(19) United States
(12) Patent Application Publication
(10) Pub. No.: US 2007/0073097 A1
(43) Pub. Date: Mar. 29, 2007

(54) System for monitoring the extracorporeal circulation and the perfusion of medical flows during cardiopulmonary bypass operations

(76) Inventors: Mirco Borra, Soliera (Modena) (IT); Corrado Bellini, Mirandola (Modena) (IT); Daniele Galavotti, Mirandola (Modena) (IT)

Correspondence Address: WILLIAM COLLARD;COLLARD & ROE, P.C., 1077 NORTHERN BOULEVARD, ROSLYN, NY, 11576, US

(21) Appl. No.: 11/526,189

(22) Filed: Sep. 22, 2006

(30) Foreign Application Priority Data
Sep. 23, 2005 (IT) MO 2005 A 000243

(51) Int. Cl. A61N 1/362

(52) U.S. Cl. 600/16; 600/504

(57) ABSTRACT

The system for the monitoring of the extracorporeal circulation and the perfusion of medical flows during cardiopulmonary bypass operations includes a substantially vertical support structure that can be associated with at least one induced circulation circuit of the blood flow in a patient and that is equipped with at least one integrated pump that supplies at least one infusion circuit that infuses a medical fluid to the patient, as it is designed to be used with means of monitoring and control of the induced circulation circuit and the infusion circuit.

The object of the invention is a system for monitoring the extracorporeal circulation and the perfusion of medical flows during cardiopulmonary bypass operations.

Current machines and systems are used for the perfusion of medical fluids and for the monitoring of the relevant therapy, before or during heart surgery with a cardiopulmonary bypass, which conventionally include a support structure in a substantially horizontal form, which is equipped with peristaltic pumps that are associated with a segment of an infusion circuit which is applied, downline, to the patient.

Such machines or systems are generally capable of the administration of medical fluids, such as those used for cardioplegia, that are able to reduce the temperature of the cardiac organ during a surgical operation.

The peristaltic pumps are applied along a segment of the infusion circuit and possess a rotor element equipped with peripheral pressure lobes that can be manually adjusted in relation to the center of rotation by a health professional according to the transverse cross-section of the tube defining the circuit itself.

The use of such machines or systems is generally supported by the use of further equipment such as circuits that are supplied by external pumps and that are able to support the extracorporeal blood circulation of the patient following the bypass of the cardiac organ.

Further equipment such as, for example, pump syringes, are conventionally used for the administration of specific medical fluids. In this case the dosage and the administration intervals are controlled by the health professional who always manually performs these procedures.

These current machines or systems present several drawbacks, including the fact that the substantially horizontal form of their support structure implies a larger blood volume in diffused extracorporeal circulation, dimensions that cannot be overlooked inside rooms, such as an operating room, that require a high degree of optimization of the space, and a possible impediment to the movements of the health professionals inside the operating room during a surgical operation.

A further drawback is the fact that the manual calibration of the positions of the lobes of the peristaltic pumps' rotor in relation to the type of pipe used is disadvantageous in terms of the amount of time spent. Furthermore, this implies the need for highly qualified personnel and does not in any case remove the existence of a margin of error due to manual calibrations that are incorrect or even considerably inexact.

The main object of the invention is to eliminate the above-mentioned drawbacks of the current state of the art by developing a system that guarantees reduced dimensions and that guarantees minimal impedance of the health professionals' movements in the operating room.

Another object of the invention is to reduce the volume of blood in extracorporeal circulation.

A further object of the invention is to guarantee the rapid adjustment of the peristaltic pumps' rotors according to the types of tubes used, thereby simultaneously reducing the margins of error during such an operation.

In the context of this technical objective, another object of the invention is to accomplish the preceding objectives with a simple structure that is relatively easy and practical to implement, that is safe to use, and that operates efficiently.

This objective and these goals are all achieved by the present system for the monitoring of the extracorporeal circulation and the perfusion of medical flows during cardiopulmonary bypass operations characterized by the fact that it includes a substantially vertical support structure that can be associated with at least one induced circulation circuit of the blood flow in a patient and that is equipped with at least one integrated pump that supplies at least one infusion circuit that infuses a medical fluid to the patient, means for monitoring and controlling of the induced circulation circuit and the infusion circuit being provided for.

Further features and advantages of the present invention will become more apparent from the detailed description of a preferred, but not exclusive, embodiment of a system for the monitoring of the extracorporeal circulation and the perfusion of medical flows during cardiopulmonary bypass operations, as specified indicatively, but not restrictively, in the combined tables of drawings in which:

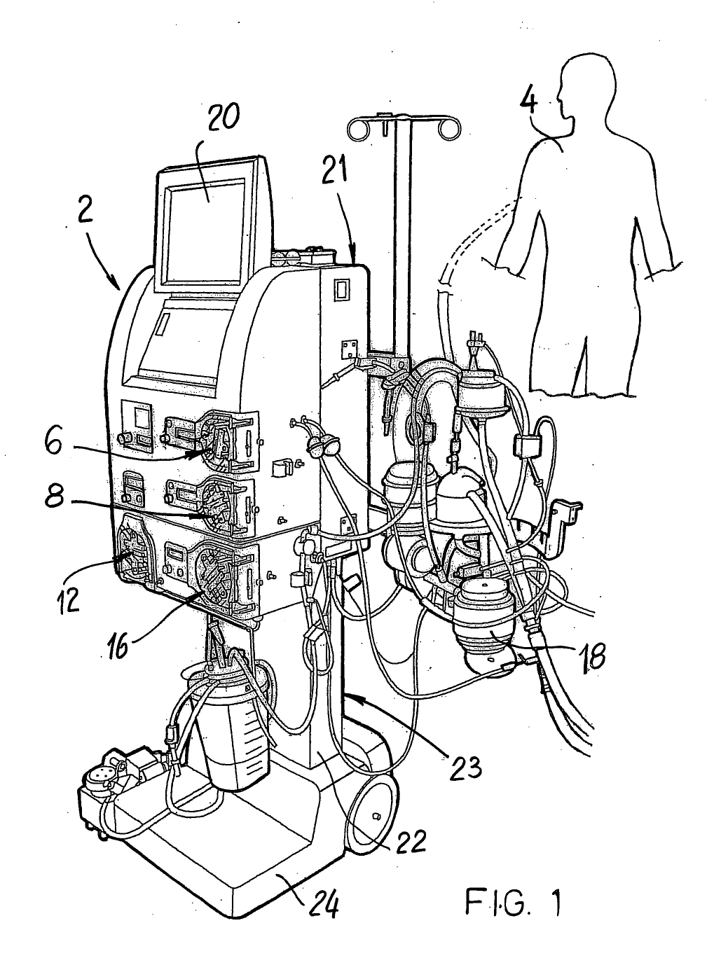

FIG. 1 is an axonometric view of the system according to the invention;

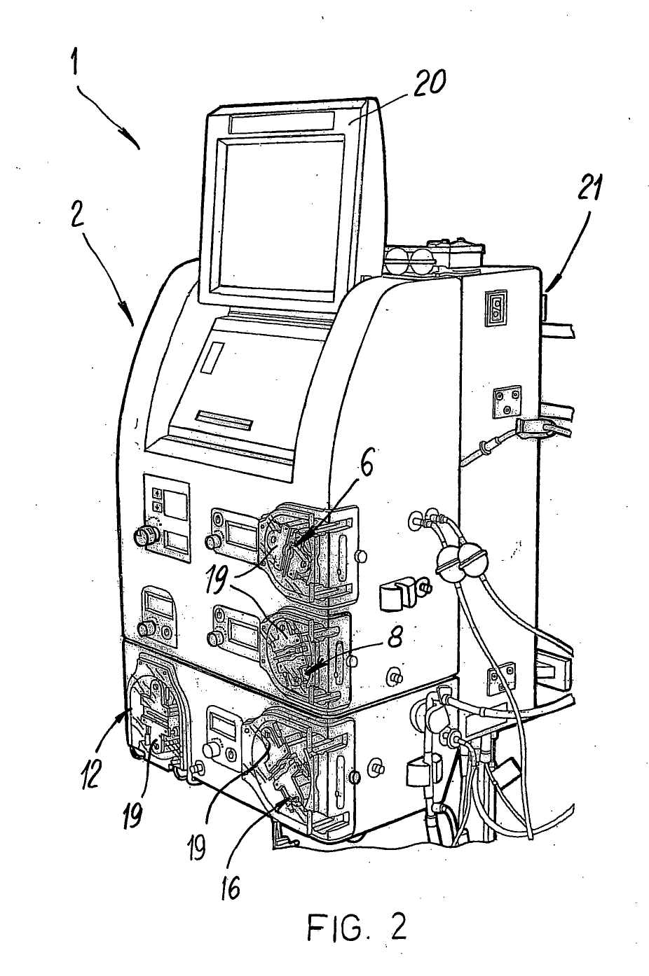

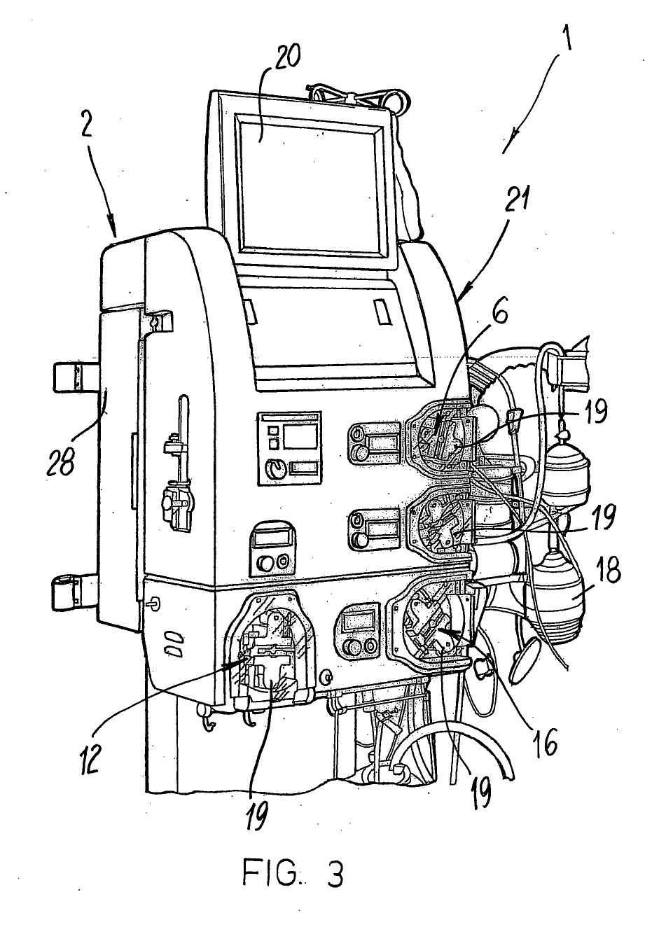

FIGS. 2 and 3 are axonometric views of a detail of the system according to the invention;

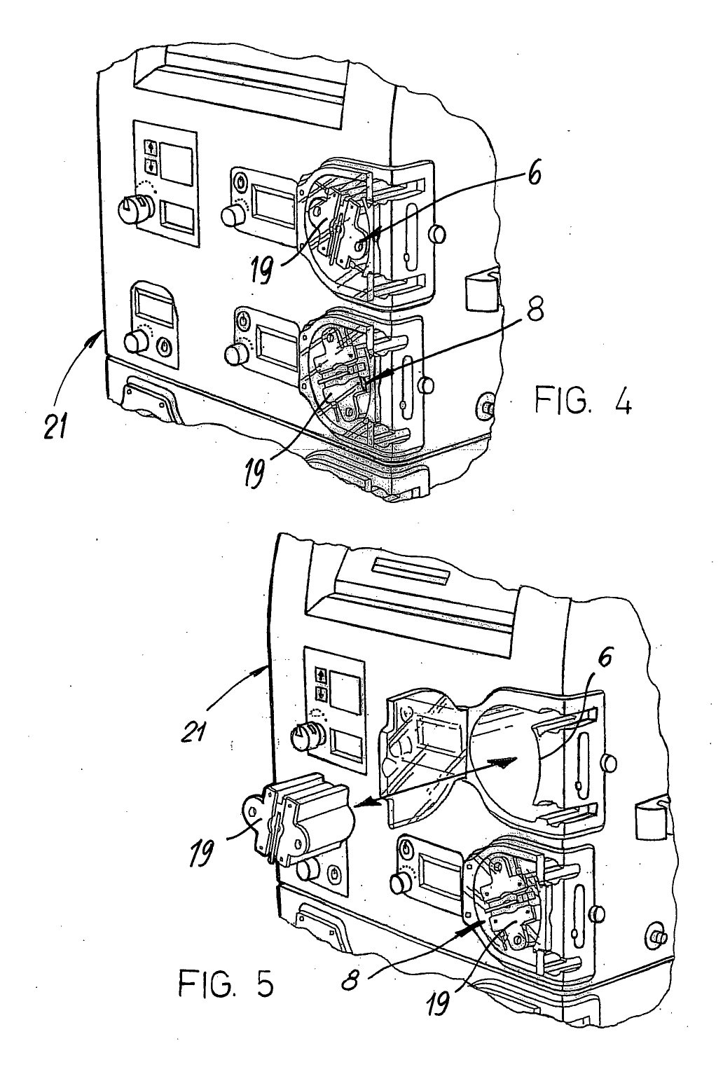

FIGS. 4 and 5 are views of a detail of the system according to the invention, which illustrates the phases of removing a pump rotor;

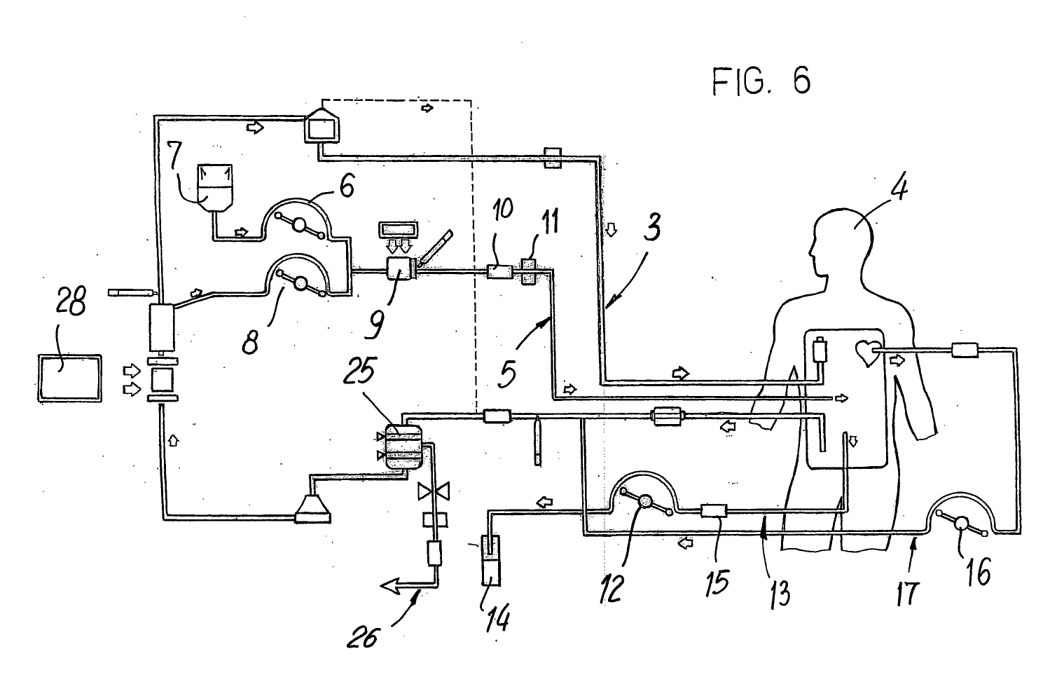

FIG. 6 is a general diagram illustrating the functional fluid screen of the system according to the invention.

With special reference to these figures, 1 is used overall to indicate the system for monitoring the extracorporeal circulation and the perfusion of medical flows during cardiopulmonary bypass operations.

The system 1 includes a substantially vertical support structure 2 that can be associated with at least one induced circulation circuit 3 of the blood flow in a patient 4 undergoing a cardiopulmonary bypass operation, and that is equipped with at least one integrated pump that supplies an infusion circuit 5 that infuses a medical fluid to the patient 4.

In detail, a first integrated pump 6 is connected to the inlet of a source of medical fluid 7, specifically, the type used for cardioplegia to cool the patient's heart during a surgical operation, while a second integrated pump 8 is connected to the inlet of the induced circulation circuit 3 for the collection of a predetermined amount of blood from the patient 4.

The blood and the medical fluid collected in this manner are mixed together according to preset amounts (for example, in relation to their respective volumes or weights) and introduced in the infusion circuit 5 connected to the respective outlets of the first and second integrated pump 6 and 8, and are perfused to the patient 4.

Alternatively the first integrated pump 6 can be connected to the inlet of the induced circulation circuit 3 and to the source of medical fluid 7, for the collection of preset amounts of blood and medical fluid, for their mixing, and for the subsequent introduction into the infusion circuit 5.

Furthermore, the infusion circuit 5 can include a cooling device 9 of the blood, a first sensor 10 for measuring the blood pressure, and a second sensor 11 for detecting air bubbles inside the circuit itself.

A third supplementary pump 12 is placed on an aspiration circuit 13 downline from the patient 4 and upline from a collection device 14.

Between the third supplementary pump 12 and the patient is placed a third pressure sensor 15 for measuring the pressure value of the aspirated blood.

The aspiration circuit 13 is used for cleaning the peritoneal zone of the patient 4 of the blood during the surgical operation, and the collection device 14 can recover the collected blood and introduce it into the induced circulation circuit 3.

A fourth supplementary pump 16 is placed on a cardiac ventilation circuit 17 and is associated downline from patient 4 and upline from the induced circulation circuit 3.

Advantageously, both the third supplementary pump 12 and the fourth supplementary pump 16 can be applied to the cardiac ventilation circuit 17 or to the aspiration circuit 13.

Also included is an external supply pump 18 for supplying the induced circulation circuit 3 that is controlled by the means of monitoring and control.

The first and the second integrated pump 6 and 8 are peristaltic type pumps that include, respectively, a rotor element 19 acting on at least one segment of the infusion circuit 5 for the propulsion of the medical fluid toward the patient 4.

The rotor element 19 is associated in a removable manner with a drive body rotated by means of removable coupling means and can be replaced with other rotors 19 that have different rotation diameters according to the dimensions of the transverse cross-section of the tubes that define the induced circulation circuit 3.

In detail, the tubes that are conventionally used in heart surgery have maximum transverse cross-section dimensions of 6.4 mm, 3.2 mm, or 9.5 mm: therefore, for each of the latter can be installed on the integrated pumps 6 and 8 a specific rotor 19 that has an external rotation diameter that is precalibrated and tested by a manufacturer in relation to the exact flow rate that the various tubes must supply, without any need to perform further manual adjustments of the positions of the lobes.

Advantageously, also the first and the second supplementary pump 12 and 16 are peristaltic type pumps that include at least one removable rotor element 19 and that act on, respectively, at least one segment of the aspiration circuit 13 and at least one segment of the ventilation circuit 17.

The means of monitoring and control include a control unit, which is not indicated in the figure for the sake of simplicity, that controls the induced circulation circuit 3 and the infusion circuit 5 and that is equipped with an interface screen 20 used for visual monitoring and for manual operations as well as setting operations, or for verification of volumes or weights, for example by means of a load cell, by a health professional.

In detail, the support structure 2 includes a box body 21 that is supported by a support 22 equipped with means of adjustment of the dimensions; the screen 19 can be folded onto the box body 21 into a closed position. The means of adjustment include a carriage that is integrally associated with said box body and guided, by means of sliding, onto a vertical support 23 that extends from a base 24.

The system 1 also includes means for detecting and removing the air from the induced circulation circuit 3, which includes at least one detecting and signaling device 25 of the presence of air that can be connected to said control unit, and aspiration means of said air that can be activated by said control unit.

In detail, the aspiration means includes a discharge circuit 26 equipped with a valve 27 that can be controlled by the control unit.

The system 1 includes means of heating and cooling the blood inside the induced circulation circuit 3 that include a heating and cooling element 28 that is placed along at least one segment of the induced circulation circuit 3.

The heating and cooling element 28 includes a plurality of Peltier cells, placed next to each other, that can be connected to an electric power supply circuit and a metal sheet that is placed between said plurality of cells and at least one segment of the induced circulation circuit 3.

Usefully, the system 1 includes means for storing the patient's physiological data and the events monitored by said control unit that consist of a CompactFlash type memory card that can be inserted in a connector that is located externally to the box body 21.

Advantageously, the machine 1 also includes an integrated printer that can be controlled by said control unit and that is able to print the values of the physiological data of the patient 4 and the events that occur during the therapy.

Furthermore, the system 1 includes a pump type syringe that can be controlled by the control unit and that can be used for the controlled administration of medical fluids.

In practice it has been verified that the described invention has achieved the proposed objects, and specifically it is underlined that the existence of a support structure with a vertical form guarantees the reduced dimensions of the system and minimal impedance of the health professionals' movements in the operating room.

Furthermore, the possibility of removing and replacing the peristaltic pumps' rotors according to the types of tubes used guarantees the rapidity of those operations for preparing the system for the surgical operation and eliminates the possibility of errors due to the incorrect calibration of the rotor.

The possibility of errors is also reduced spontaneously, and in any case identifiably, due to the existence of means of storage of all the events and the patient's conditions during the operation and the comparison of these data with the desired events and conditions.

The monitoring of the induced circulation circuit and the introduction circuit by means of a single screen, together with the possibility of monitoring and controlling other instruments, such as a pump syringe, makes it possible to limit errors due to confusion by the health professionals, thereby also increasing the speed of the actions taken by the health professionals themselves under emergency conditions.

The invention conceived in this manner is subject to numerous modifications and variations, all of which are comprised in the context of the inventive concept. Furthermore, all the details can be replaced with other elements that are technically equivalent.

In practice the materials used, as well as the corresponding forms and dimensions, can be varied to any degree according to requirements, without this causing said variations to fall outside the purview of the protection of the following claims.

1. System for the monitoring of the extracorporeal circulation and the perfusion of medical flows during cardiopulmonary bypass operations comprising a substantially vertical support structure that can be associated with at least one induced circulation circuit of the blood flow in a patient and that is equipped with at least one integrated pump that supplies at least one infusion circuit that infuses a medical fluid to said patient, means of monitoring and control of said induced circulation circuit and said infusion circuit being provided for.

2. System according to claim 1, wherein said at least one integrated pump is a peristaltic type pump that includes at least one precalibrated rotor element acting on at least one segment of said infusion circuit for the propulsion of said medical fluid toward said patient, a drive body rotated by said precalibrated rotor element, and means of removable coupling of said precalibrated rotor element to said drive body.

3. System according to claim 1, wherein the integrated pump can be associated with the inlet of said induced circulation circuit and at least one source of said medical fluid and that can be associated with the outlet of said infusion circuit.

4. System according to claim 1, wherein the integrated pump comprises a first integrated pump that can be associated with the inlet of at least one source of said medical fluid and a second integrated pump that can be associated with the inlet of said induced circulation circuit, as said first and second integrated pump can be associated with the outlet of said infusion circuit for the mixing of said fluid and of said blood.

5. System according to claim 1, comprising a third supplementary pump that is placed on a blood aspiration circuit downline from said patient and upline from a collection device.

6. System according to claim 5, comprising a fourth supplementary pump that is placed on a cardiac ventilation circuit of said patient and that can be associated downline from said patient and upline from said induced circulation circuit.

7. System according to claim 5 wherein said third supplementary pump and said fourth supplementary pump are peristaltic type pumps and comprise at least one precalibrated rotor element acting, respectively, on at least one segment of said aspiration circuit and on at least one segment of said ventilation circuit, a drive body rotated by said precalibrated rotor element, and means of removable coupling of said precalibrated rotor element to said drive body.

8. System according to claim 1, wherein said means of monitoring and control comprises a control unit installed on said support structure.

9. System according to claim 8, wherein said control unit can be associated with at least one external pump that supplies said induced circulation circuit.

10. System according to claim 1, comprising means of detecting and removing the air from said induced circulation circuit.

11. System according to claim 10, wherein said means of detecting and removing includes at least one device for detecting and signaling the presence of air that can be connected to said control unit, and means of aspiration of said air that can be activated by said control unit.

12. System according to claim 1, comprising means of heating and cooling the blood contained in said induced circulation circuit.

13. System according to claim 12, wherein said means of heating and cooling include a heating and cooling element that is placed along at least one segment of said induced circulation circuit.

14. System according to claim 13, wherein said heating and cooling element comprises at least one Peltier cell that can be connected to an electric power supply circuit.

15. System according to claim 13 wherein said heating and cooling element comprises a plurality of said Peltier cells placed next to each other, with at least one metal sheet placed between said plurality of cells and said at least one segment of said induced circulation circuit.

16. System according to claim 1 comprising means of storing the patient's physiological data and the events monitored by said control unit.

17. System according to claim 16, wherein said means of storing comprises a memory card.

18. System according to claim 1 comprising at least one integrated printer that can be controlled by said control unit.

19. System according to claim 1 comprising a pump type syringe that can be controlled by said control unit for the controlled administration of said medical fluids.

20. System according to claim 1, wherein said support structure comprises a box body that is supported by a support equipped with means of adjustment of the dimensions.

21. System according to claim 20, wherein said means of adjustment of the dimensions comprises a carriage that is integrally associated with said box body and guided, by means of sliding, onto a vertical support that extends from a base.

22. System according to claim 1, comprising a screen connected to said control unit, that is contact-activated and that can be folded onto said box body into a closed position.
